(19) United States
(12) Patent Application Publication
(10) Pub. No.: US 2004/0127771 A1
(43) Pub. Date: Jul. 1, 2004

(54) Diagnostic instrument

(75) Inventors: Helmut A. Heine, Herrsching (DE); Norbert Merkt, Breitbrunn (DE)

Correspondence Address: ROTHWELL, FIGG, ERNST & MANBECK, P.C., 1425 K STREET, N.W., SUITE 800, WASHINGTON, DC, 20005, US

(73) Assignee: Heine Optotechnik GmbH & Co. KG, Herrsching (DE)

(21) Appl. No.: 10/734,219

(22) Filed: Dec. 15, 2003

(30) Foreign Application Priority Data
Dec. 27, 2002 (DE) 202 19 984.3

(51) Int. Cl. A61B 1/06; A61B 1/267

(52) U.S. Cl. 600/200; 600/179

(57) ABSTRACT

A diagnostic instrument, in particular an otoscope (1), including a body with a funnel-shaped housing (2), and a base (6) formed integrally on the housing (2), and lighting arranged in the housing or in the base (6). The lighting has a light-emitting diode (10), and the body accommodates adapter electronics (11) which adapt voltage delivered from outside to a voltage required by the light-emitting diode (10).

### BACKGROUND OF THE INVENTION

1. Field of the Invention

The invention relates to a diagnostic instrument, in particular an otoscope with a lighting means for illuminating a patient's inner ear. Other instruments in question here are, for example, pharyngoscopes, mouth mirrors, diascleral transilluminators, and transcutaneous lamps.

2. Description of the Background Art

A known otoscope 1 comprising a body whose housing 2 is substantially in the shape of a funnel is shown in cross section in FIG. 1. The wider end of the funnel-shaped housing 2 directed toward the observer permits viewing, via an electric light bulb 4, into the narrowed distal end 5 of the otoscope 1 and onward into the ear of the patient. A tubular attachment piece or base 6 is formed integrally on the funnel-shaped housing 2. The otoscope 1 sits with its base 6 on a universal handgrip (not shown) which accommodates the batteries or accumulators for supplying the electric light bulb 4 with current via a current conductor 7.

In FIG. 2, an otoscope is shown which is of the same design externally but in which the light from the electric light bulb 4 arranged in the base 6 is conveyed to the distal end 5 of the otoscope 1 via a fiber-optic guide 8. The fiber-optic bundle 8 is cylindrical near the end directed toward the light bulb 4 and widens out in a tube shape toward the distal end 5 of the otoscope 1. An inner funnel 9 serves to support the fiber-optic bundle 8 in the area of its tube-shaped widening.

Conventional otoscopes with conventional electric light bulbs as lighting means have a number of disadvantages. To ensure that the field to be lit is illuminated as intensely as possible, the electric light bulbs are operated with a relatively high voltage, with the result that they have only a short working life. Because of the high current demand, the associated batteries or accumulators also quickly run dry. The otoscope 1 shown in FIG. 1 and working with direct illumination also has the disadvantage that the relatively large electric light bulb 4 impedes the view into the distal end of the housing 2, and the heat radiated by the electric light bulb is at the very least dicomforting for the patient, and, in the worst case, can cause pain and injuries.

### SUMMARY OF THE INVENTION

The object of the invention is therefore to equip an otoscope with a longer-lasting lighting means which, while providing a comparable light intensity, has a longer working life and consumes less current, and radiates less heat, than is the case with conventional electric light bulbs.

To achieve this object, it is proposed to use a light-emitting diode (LED) as lighting means instead of a conventional electric light bulb. These LEDs have an almost unlimited working life and a low current consumption, and they emit negligible heat radiation. However, they require other voltages than those of electric light bulbs usually used for otoscopes and other diagnostic instruments. The design of the universal handgrip would therefore have to be changed, but it would then lose its ability to accommodate and supply other medical diagnostic instruments too. According to the invention, therefore, in addition to the light-emitting diode, adapter electronics are also arranged in the otoscope itself.

### BRIEF DESCRIPTION OF THE DRAWINGS

The prior art and the invention are explained in more detail below with reference to the attached drawings, in which:

FIG. 1 shows a diagrammatic cross section of a known otoscope with direct illumination,

FIG. 2 shows a diagrammatic cross section of a known otoscope with indirect illumination via a fiber-optic bundle,

FIG. 3 shows a diagrammatic cross section of an otoscope according to the invention with direct illumination, and

FIG. 4 shows a diagrammatic cross section of an otoscope according to the invention with indirect illumination.

### DETAILED DESCRIPTION OF THE INVENTION

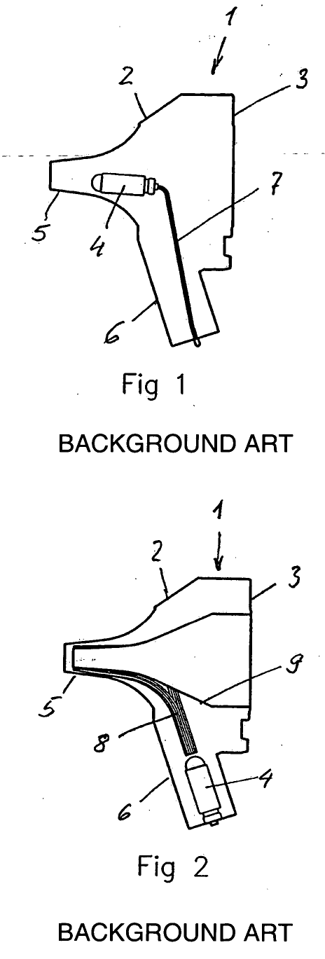

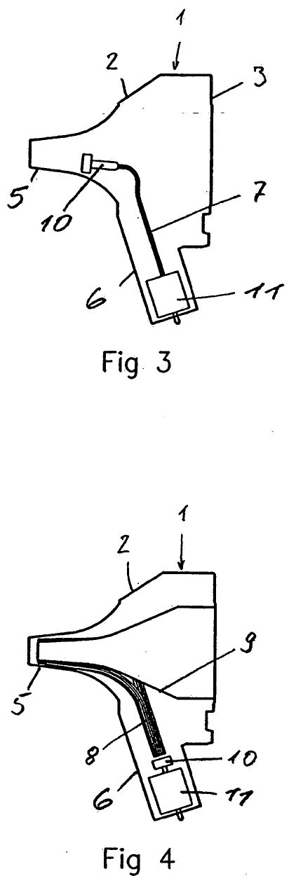

FIGS. 3 and 4 show otoscopes 1 designed according to the invention with a light-emitting diode 10 and adapter electronics 11. In the direct otoscope shown in FIG. 3, the light-emitting diode 10 is situated, as in the embodiment in FIG. 1, in the narrowed part of the housing 2, there taking up considerably less space than the electric light bulb 4 in the otoscope 1 in FIG. 1. It therefore does not impede the view into the patient's ear.

In the indirectly illuminated otoscope 1 shown in FIG. 2, the light-emitting diode 10 and the adapter electronics 11 sit together in the base 6 of the otoscope 1.

In a further embodiment not shown here, the light-emitting diode 10 and adapter electronics 11 are integrated to form a single component whose dimensions correspond mechanically and electrically to a conventional electric light bulb. In this way, in an otoscope of the type shown in FIGS. 1 and 2, the electric light bulb 4 can be replaced by this integrated component. It is then unnecessary to procure a new otoscope according to FIGS. 3 and 4. The otoscope 1 in FIG. 1 when equipped with the integrated component has the advantage that a light-emitting diode creates much less heat, and the patient is therefore not inconvenienced by heat.

In a further preferred embodiment, the transparent part of a housing of the light-emitting diode 10 is provided with an optical system, for example a lens-shaped dome, so that the field to be illuminated can be uniformly lit. This also applies when using a fiber-optic guide for conveying the light from a light-emitting diode arranged in the base.

1. A diagnostic instrument comprising a body including a funnel-shaped housing and a base formed integrally on the housing, lighting means being arranged in the body, wherein said lighting means comprises a light-emitting diode, and said body accommodates adapter electronics which adapt voltage delivered from outside to a voltage required by said light-emitting diode.

2. The diagnostic instrument as claimed in claim 1, wherein said light-emitting diode and said adapter electronics are integrated in one component which corresponds mechanically and electrically to a conventional electric light bulb.

3. The diagnostic instrument as claimed in claim 1, wherein said said light-emitting diode comprises a transparent housing on which an optic is formed integrally.
